(19) United States
(12) Patent Application Publication
(10) Pub. No.: US 2025/0161385 A1
(43) Pub. Date: May 22, 2025

(54) FOOD SUPPLEMENT

(71) Applicants: Mikhail Ankirsky, San Francisco, CA (US); Konstantin Sotnikov, Almaty (KZ)

(72) Inventors: Mikhail Ankirsky, San Francisco, CA (US); Konstantin Sotnikov, Almaty (KZ)

(21) Appl. No.: 18/810,726

(22) Filed: Aug. 21, 2024

(30) Foreign Application Priority Data
Nov. 20, 2023 (RU) W23066879

(51) Int. Cl. A61K 36/05; A61K 9/00

(52) U.S. Cl. CPC A61K 36/05; A61K 9/0053; A61K 2236/11; A61K 2236/51; A61K 2236/53

(57) ABSTRACT

A feed additive for farm animals and poultry is provided, including biomass and/or cultural liquid of the microalgae Chlorella vulgaris together with chlorellin in a titer of 108-1014 CFU per 1 ml of cultural liquid and/or biomass. Related methods are also provided.

### CROSS-REFERENCE TO RELATED APPLICATIONS

This application claims priority to Russian Application No. 2023130074, having a filing date of Nov. 20, 2023, the entire contents of which are hereby incorporated by reference.

### FIELD OF TECHNOLOGY

The following relates to feed production and a feed additive for farm animals and poultry, including biomass and/or cultural liquid of the microalgae Chlorella vulgaris together with chlorellin in a titer of 10⁸-10¹⁴ CFU per 1 ml of cultural liquid and/or biomass.

The following makes it possible to increase the productivity of farm animals and poultry by 7-15% compared to known feed additives containing Chlorella.

### BACKGROUND

Embodiments of the invention relate to feed production and a feed additive containing biomass and/or cultural liquid obtained by cultivating a strain of green unicellular microalgae Chlorella vulgaris, mixed with the probiotic composition Chlorellin.

Chlorella has been used in animal husbandry for a very long time. Chlorella suspension has a complex effect on the animal body. The suspension contains all amino acids, vitamins, prebiotic components, immune stimulants, which has a positive effect on the animal's body.

The effectiveness of using Chlorella is described in various sources of information, for example, in the article by Belan N. I. “The effectiveness of using Chlorella in poultry nutrition” Agricultural Journal, vol. 1, no. 8, 2015, pp. 20-22.

Document RU2764917 describes a method for increasing the productivity of broiler chickens, characterized by the fact that broiler chickens include in the main diet the lactulose-containing additive Lactuvet-1 at a dose of 0.4 g/kg of live weight in the age period from 1 to 38 days or the lactulose-containing additive Lactuvet-1. 1 at a dose of 0.4 g/kg live weight and a suspension of Chlorella at a dose of 5 ml/kg of feed.

From document RU2501295, a method for producing a feed additive for animals containing Chlorella is known, including mixing pre-treated cellulose-containing raw materials with nutritional additives, introducing microorganisms, aging and subsequent processing, characterized in that sunflower husks and beet pulp are used as cellulose-containing raw materials, pre-treatment of the cellulose-containing raw materials are extruded at a temperature of 110-130° C., the resulting extrudate is crushed, followed by 5-10 minutes of stirring and adding nutritional supplements in the form of beer wort and a Chlorella suspension, with the initial components taken in the following ratio, wt. %:
beet pulp 15-20
sunflower husk 75-77.5
beer wort 0.3-0.5
Chlorella suspension the rest,
then microorganisms Trichoderma viride pcs. are added to the resulting mixture. F-98 in an amount of 1-2% by weight of the mixture and kept for 6-8 days at a temperature of 26-30° C. with periodic stirring, the resulting wet mixture is dried to a moisture content of 10-12% at a temperature of 40-50° C. and crushed.

From document RU2538399, a product of processing a suspension of planktonic strains of Chlorella is known, which has biological activity and is a concentrate of Chlorella cell biomass with a cell density of 30 to 1000 million/ml, or a concentrate of the culture medium of Chlorella cells. This product is used in animal feed.

From document RU2708161, a complex biologically active feed additive for animals and birds is known, characterized in that it contains zeolite powder, a probiotic, including biomass of lactic acid bacteria selected from Lactobacillus acidophilus, or Propionobacterium freundenreichii, or Bifidobacterium longum, or Bifidobacterium adolescentis, or Streptococcus thermophiles with a titer of at least 109 CFU/ml, freshwater microalgae Chlorella, feed glucose and cereal bran. The entire contents of RU2708161 are hereby incorporated by reference.

However, despite the widespread use of Chlorella in feed production, there is a need for effective preparations, namely, feed additives containing Chlorella, which can increase the productivity and natural resistance of farm animals and poultry, compensate for the deficiency of amino acids, vitamins, microelements in the diet, and increase the digestibility of feed, normalize the microflora of the gastrointestinal tract.

### SUMMARY

An aspect relates to an effective biologically active feed additive using a simple and waste-free production technology that allows the use of not only microalgae biomass, but also cultural liquid

The technical result of embodiments of the invention is to increase the physiological and immune status of the body of animals and poultry, compensate for the deficiency of amino acids, vitamins and microelements in the feeding diet, increase the digestibility of feed and stimulate weight gain, and as a result, increase productivity, and productivity increases by 20-25% compared to the control without Chlorella additives and by 7-15% compared to the closest analogue containing Chlorella in its composition.

The technical result is achieved by creating a feed additive for farm animals and poultry, including biomass and/or cultural liquid of Chlorella vulgaris together with chlorellin in a titer of 10⁸-10¹⁴ CFU per 1 ml of cultural liquid and/or biomass. Thus, the combination of biomass and/or culture liquid of Chlorella vulgaris together with chlorellin in a titer of 10⁸-10¹⁴ CFU per 1 ml of culture liquid and/or biomass as a feed additive has an unexpected effect that makes it possible to increase the productivity of farm animals and birds.

The possibility of combining biomass and/or culture liquid of Chlorella vulgaris with chlorellin in a titer of 10⁸-10¹⁴ CFU per 1 ml of culture liquid and/or biomass as a feed additive is unknown from the conventional art, and it is also not obvious that their combination leads to such a significant increase in the productivity of farm animals birds by 7-15% compared to known feed additives containing Chlorella.

The essence of embodiments of the invention is as follows: the proposed feed additive as a complex of biologically active substances contains biomass and/or cultural liquid obtained by cultivating a strain of green unicellular microalgae Chlorella vulgaris in a mixture with the substance chlorellin. Chlorellin is a substance with natural antibiotic activity and is produced by Chlorella. In this case, chlorellin is contained in the additive in a titer of 10⁸-10¹⁴ colony-forming units (CFU) per 1 ml of culture liquid and/or microalgae biomass.

In embodiments, the method of raising animals and poultry involves introducing a feed additive into the normal diet of young animals and poultry with feed or drinking water. The feed additive is introduced into the diet of animals and poultry, starting from one day of age.

The feed additive is based on a strain of green unicellular microalgae Chlorella vulgaris.

Chemical composition of the biomass of the green unicellular microalgae Chlorella vulgaris:

### Content

| | |
|---|---|
| crude protein | 47-54% |
| carbohydrates | 23-26% |
| including glycans | 16-20% |
| lipids | 7-9% |
| nucleic acids | 4-6% |
| minerals | 8-10% |
| vitamins (E, H, group B) | 0.03-0.05% |
| amino acids, (% of asv) | 27-30% |
| microelements | 1.5-3% |

### Vitamin Composition of Biomass:

| A (retinol) | Not detected | |
|---|---|---|
| B (tocopherol) | 32.0-36.0 | mg/kg |
| B1 (thiamine chloride) | 1.3-3.5 | mg/kg |
| B2 (riboflavin) | 83.5-87.8 | mg/kg |
| B3 (pantothenic acid) | 141.0-143.5 | mg/kg |
| B5 (nicotinic acid) | 71.4-76.9 | mg/kg |
| B6 (pyridoxine) | 8.0-8.4 | mg/kg |
| Sun (folic acid) | 11.8-13.2 | mg/kg |
| B12 (cyanocobalamin) | 0.1-0.4 | mg/kg |
| H (biotin) | 0.8-1.5 | mg/kg |

### Content of Microelements in Biomass, Mg/Kg:

| | |
|---|---|
| potassium | 13150-13240 |
| sodium | 5370-5650 |
| calcium | 1900-1925 |
| magnesium | 1205-1234 |
| iron | 780-900 |
| copper | 21.8-23.4 |
| zinc | 59.1-60.5 |
| manganese | 209-233 |
| chrome | 8.0-8.4 |
| nickel | 6.5-7.4 |
| lead | 0.5-1.0 |
| cadmium | Not detected |
| arsenic | 0.01 |

### Composition and Amino Acid Content of Biomass Protein, %:

| | |
|---|---|
| lysine | 0.63-0.98 |
| histidine | 1.0-1.17 |
| arginine | 0.1-1.01 |
| aspartic acid | 1.8-2.17 |
| threonine | 0.21-0.33 |
| serine | 0.15-0.23 |
| glutamic acid | 2.05-2.67 |
| proline | 1.9-1.19 |
| glycine | 0.8-1.05 |
| Alanine | 1.0-1.24 |
| cystine | 0.19-0.38 |
| valine | 0.9-1.5 |
| methionine | 0.12-0.22 |
| isoleucine | 0.9-1.37 |
| leucine | 1.8-2.18 |
| tyrosine | 0.3-0.81 |
| phenylalanine | 1.2-1.51 |

Chemical Composition of the Culture Liquid of the Green Unicellular Microalga Chlorella vulgaris:

### General Analysis:

| | |
|---|---|
| Dry matter | 1.5% |
| Organic acids | 10-12 g/l |
| Fatty acids | 2.5 g/l |

### Microelements, Mg/l:

| | |
|---|---|
| K | 800-1000 |
| Na | 300-400 |
| Ca | 200-300 |
| N | 130-200 |
| Mg | 30-40 |
| Fe | 0.1-0.7 |
| Cu | 0.0-0.05 |
| Zn | 0.4-0.6 |
| Mn | 0.2-0.07 |
| Ni | 0.001-0.003 |
| Co | 0.003-0.008 |
| Rv | Not found |
| Pg | Not found |
| As | Not found |

### Composition and Content of Amino Acids in the Culture Liquid:

| B1 | 0.04-0.09 | mg/l |
|---|---|---|
| B2 | 1.0-1.2 | mg/l |
| B3 | 0.5-0.7 | mg/l |
| B4 | Not detected | |
| B5 | 95.0-97.0 | mg/l |
| B6 | 0.4-0.6 | mg/l |
| B9 | 0.19-0.25 | mg/l |

| | |
|---|---|
| B12 | Not found |
| H | Not sufficient |

### Vitamin Composition of the Culture Liquid:

| B1 | 0.09 | mg/l |
|---|---|---|
| B2 | 1.0-1.2 | mg/l |
| B3 | 0.5-0.7 | mg/l |
| B4 | not found | |
| B5 | 95.0-97.0 | mg/l |
| B6 | 0.4-0.6 | mg/l |
| B9 | 0.19-0.25 | mg/l |

| | |
|---|---|
| B12 | not found |

### Amount and Composition of the Organic Acid Fraction in the Culture Fluid:

| | |
|---|---|
| citric acid | 0.4-0.8 g/l |
| fumaric acid | 0.3-0.6 g/l |
| malic acid | 0.3-0.5 g/l |
| oxalic-acetic acid | 0.4-0.9 g/l |

The introduction into the feed of farm animals and poultry of a feed additive containing biomass and/or cultural liquid obtained by cultivating the green unicellular microalgae Chlorella vulgaris, or biomass in combination with the probiotic substance Chlorellin, helps to increase the level of natural resistance of the body, reduce the risk of developing diseases, and activates the process digestion due to the normalization of the composition and functional activity of the intestinal microflora, which leads to increased productivity of farm animals and poultry.

In embodiments, the method of obtaining a feed additive consists of growing a strain of green unicellular microalgae Chlorella vulgaris in a nutrient medium, separating the culture medium into culture liquid and biomass, and concentrating them. The biomass and culture liquid are standardized, packaged and packaged. The biomass and culture liquid can be pre-dried by any known method, such as freeze-drying or spray drying.

There are three options for feed additives:
1) in the form of microalgae biomass Chlorella vulgaris in combination with Chlorellin,
2) in the form of culture liquid Chlorella vulgaris in combination with Chlorellin,
3) and in the form of a mixture of biomass and culture liquid of Chlorella vulgaris, in combination with Chlorellin.

The biomass of the green unicellular microalgae Chlorella vulgaris is grown on a rocking chair at a temperature of 30° C. and pH 5.5 in a nutrient medium.

When grown under conditions of intensive aeration and mixing of the environment, the accumulation of biomass of the green unicellular microalga Chlorella vulgaris occurs after 36-48 hours.

The grown biomass is separated from the culture liquid. It is possible to concentrate the resulting products.

The biomass and culture liquid can be dried followed by granulation.

Chlorellin is added in an amount of 10⁸-10¹⁴ colony forming units (CFU) per 1 ml of culture liquid and/or microalgae biomass.

After quality analysis, the feed additive is packaged and packaged.

The effectiveness of feed additives is assessed by the increase in average live weight of farm animals and poultry;

### EXAMPLE 1

### Preparation of Feed Additive According to Embodiments of the Invention.

The biomass of the green unicellular microalgae Chlorella vulgaris strain IFR No. C-111 is grown on a rocking chair at a temperature of 30° C. and pH 5.5 in Tamiya nutrient medium.

When grown under conditions of intensive aeration and mixing of the environment, the accumulation of biomass of the green unicellular microalga Chlorella vulgaris occurs after 36-48 hours.

The grown biomass is separated from the culture liquid. It is possible to concentrate the resulting products.

The biomass and culture liquid can be dried followed by granulation.

Chlorellin is added in an amount of 10⁸-10¹⁴ colony forming units (CFU) per 1 ml of culture liquid and/or microalgae biomass.

After quality analysis, the feed additive is packaged and packaged.

The effectiveness of feed additives is assessed by the increase in average live weight of farm animals and poultry;

### EXAMPLE 2

### Preparation of Feed Additive According to Embodiments of the Invention.

The biomass of the green unicellular microalgae Chlorella vulgaris strain IFR No. C-111 is grown on a rocking chair at a temperature of 30° C. and pH 5.5 in Tamiya nutrient medium.

When grown under conditions of intensive aeration and mixing of the environment, the accumulation of biomass of the green unicellular microalga Chlorella vulgaris occurs after 36-48 hours.

After 48 hours, the amount of biomass of the green unicellular microalgae Chlorella vulgaris was 8 g/l.

The grown biomass is separated from the culture liquid by filtration on a suction filter.

The protein yield in the resulting biomass is 54%.

Thus, three product variants were obtained. One option contains only Chlorella vulgaris biomass, the second—Chlorella vulgaris culture liquid, the third—a mixture of culture liquid and Chlorella vulgaris biomass.

The substance chlorellin was added to each of the variants of the resulting products in an amount of 10⁸ colony-forming units (CFU) per 1 ml of culture liquid and/or microalgae biomass.

Each of the resulting additive variants was dried using freeze drying.

Example 2 is carried out similarly to Example 1, only to obtain variants of the feed additive, chlorellin is added to the biomass and/or culture liquid in an amount of 10¹⁴ colony forming units (CFU) per 1 ml of culture liquid and/or microalgae biomass.

Each of the resulting additive variants was dried using freeze drying.

### EXAMPLE 3

### Checking the Effectiveness of the Resulting Feed Additive Options.

The formation of experimental groups of calves was carried out in accordance with the methodology for conducting zootechnical experiments (A. I. Ovsyannikov, 1976). To conduct scientific and economic experiments to determine the effectiveness of feeding KD, groups of calves (bulls) of the black and white breed were formed using the method of pairs of analogues, taking into account origin, age, live weight and health status.

Calves of the compared groups received feed according to the calf feeding scheme used on the farm. At the same time, calves of the second experimental group additionally received a feed additive with drinking water in free access throughout the day. To control the completeness of feeding and metabolic processes in calves, the morphological and biochemical parameters of their blood were studied using generally accepted methods (Lebedev P. T. and Usovich A. T., 1976; Kondrakhin I. P., 2004). To determine the average daily gain in live weight, the animals were weighed in the morning before feeding every 10 (ten) days and the absolute gain was determined when removed from the experiment.

The duration of the experiment was 48 days (Table 1).

TABLE 1
Scheme of scientific and economic experiments on calves

| | Number | | Study | |
|---|---|---|---|---|
| Group of | of | | period, | Feeding |
| animals | animals | Breed | days | conditions |
| I-control | 20 | Black-motley | 48 | Basic diet (BR) |
| II- | 60 | Black-motley | 48 | OR + feed additive |
| experimental | | | | drinking H2 O for 1 |
| group using | | | | head/day |
| embodiments | | | | |
| III- | 20 | Black-motley | 48 | OR + feed additive |
| experimental | | | | according to |
| group using | | | | RU2708161 |
| RU2708161 | | | | |

Animals in the control group without supplementation received the basic diet. And the calves of the second experimental group, in addition to the main diet, received the tested feed additive, introduced in the composition with drinking water. At the same time, animals of the third experimental group with an additive from RU2708161 received the main diet with the specified additive, as described in the patent, experiment No. 3 (main diet+dietary supplement set 1%).

The feeding rations of the experimental animals and their housing conditions were the same, i.e. were in a standard room, stall—walking housing. Watering was done from drinking bowls, feeding was carried out from feeders. Silage, haylage and hay were distributed in the form of feed mixtures, twice using a feed dispenser of the “Host” type. In addition, the calves ate hay and straw also on the card in free access.

The results of studies of the chemical composition and calculations of the nutritional value of basic feeds showed that the feeds used in feeding experimental animals met zootechnical and physiological needs.

The diet for feeding the calves consisted of rump hay—1.5 kg, corn silage—14 kg, grain mixture (barley—5.5 kg, crushed corn—0.5 kg), additives (chalk—0.08 kg, table salt—0.08 kg, premix P63-1 mol.—0.07 kg), dried wheat stillage 34.9%—1.5 kg.

At the same time, 20 animals of the experimental group received a supplement consisting of biomass of the Chlorella vulgaris strain and chlorellin in an amount of 10⁸ colony-forming units (CFU) per 1 ml of culture fluid according to embodiments.

Another 20 animals in the experimental group received a supplement consisting of a culture liquid of the Chlorella vulgaris strain and chlorellin in an amount of 10¹⁴ colony-forming units (CFU) per 1 ml of culture liquid according to embodiments.

And another 20 animals in the experimental group received a supplement consisting of culture liquid and biomass of the Chlorella vulgaris strain and chlorellin in an amount of 10¹⁰ colony-forming units (CFU) per 1 ml of culture liquid according to embodiments.

The amount of additive added was 1 liter for 12 500 liters of fresh water.

Better digestibility of nutrients and their absorption of mineral and nitrogenous substances from the diet by calves ensured an increase in the average daily gain of their live weight and a decrease in feed consumption to obtain 1 kg of gain. The growth rate of calves when using the feed additive is given in Table 2.

TABLE 2
Growth rate of calves, kg

| | Group | | |
|---|---|---|---|
| | I- | II- | III- |
| Index | control* | experimental* | experimental* |
| Live weight at the | 257.73 | 315.88 | 301.38 |
| beginning of the | | | |
| experiment: | | | |
| at the end of the | 309.27 | 380.31 | 358.27 |
| experiment | | | |
| Gain | 51.54 | 64.43 | 56.89 |
| Absolute increase | 100 | 125.0 | 110 |
| In % to control | | | |
| Average daily increase | 1.073 | 1.342 | 1.185 |
| In % to control | 100 | 125.0 | 110 |

*Values were calculated as the arithmetic mean of all animals in the group.

At the end of the experiment, the absolute increase in live weight in the second experimental group was greater by 12.89 kg, or 25.0% compared to animals in the control group. At the same time, the average daily increase in live weight in the control group was 1.073 kg, and in the experimental group—1.342 kg, or 25.0% more compared to animals in the control group. It should be noted that the results in the III experimental group were lower than in the II experimental group. Therefore, it can be concluded that the feed additive according to embodiments of the invention are more effective than the feed additive according to that reference.

At the same time, the studies established that by the end of the experiment, the animals of the second experimental group were significantly different from their peers from the control group, they were more mobile, their fur was elastic, shiny, and their skin looked cleaner than in the control. The use of a feed additive with drinking water had a positive effect on metabolic processes, feed palatability, digestibility and assimilability of diet nutrients, which contributed to an increase in live weight gain.

Experiments with poultry were carried out in a similar way. For the experiments broiler chickens of the Cobb-500 cross were chosen.

Chickens of the compared groups received feed according to the feeding schedule. At the same time, the chickens of the second experimental group additionally received a feed additive with drinking water in free access throughout the day. To determine the average daily gain in live weight, the animals were weighed in the morning before feeding every 10 (ten) days and the absolute gain was determined when removed from the experiment.

The duration of the experiment was 30 days (from 8 to 38 days) (Table 3).

TABLE 3
Scheme of scientific and economic experiments on broiler chickens

| | | | Study | |
|---|---|---|---|---|
| Group of | Number of | | period, | |
| animals | animals, n | Breed | days | Feeding conditions |
| I-control | 20 | cross Cobb- | 30 | Basic diet (BD) |
| group | | 500 | | |
| II- | 60 | cross Cobb- | 30 | BD + feed additive |
| experimental | | 500 | | with drinking |
| group using | | | | H2 O for 1 head/day |
| embodiments | | | | |
| III- | 20 | cross Cobb- | 30 | BD + feed |
| experimental | | 500 | | additive according |
| group using | | | | to RU2708161 |
| RU2708161 | | | | |

Animals in the control group received the basic diet. And the broiler chickens of the experimental group, in addition to the main diet, received the tested feed additive, introduced in the composition with drinking water. At the same time, animals of the third experimental group with an additive from RU2708161 received the main diet with the specified additive as described therein, experiment No. 3 (main diet+dietary supplement set 1%).

The feeding rations of the experimental animals and their living conditions were the same.

The results of studies of the chemical composition and calculations of the nutritional value of basic feeds showed that the feeds used in feeding experimental animals met zootechnical and physiological needs.

### Feeding Diet for Broiler Chickens:

50% corn;
20% wheat;
20% sunflower cake;
10% BMVD, i.e. protein-mineral vitamin supplement.

At the same time, 20 animals of the experimental group received a supplement consisting of biomass of the Chlorella vulgaris strain and chlorellin in an amount of 10⁸ colony-forming units (CFU) per 1 ml of culture fluid.

Another 20 animals in the experimental group received a supplement consisting of a culture liquid of the Chlorella vulgaris strain and chlorellin in an amount of 10¹⁴ colony-forming units (CFU) per 1 ml of culture liquid.

And another 20 animals in the experimental group received a supplement consisting of culture liquid and biomass of the Chlorella vulgaris strain and chlorellin in an amount of 10¹⁰ colony-forming units (CFU) per 1 ml of culture liquid.

The amount of additive added was 1 liter per 12 500 liters of fresh water.

The growth rate of chickens when using the feed additive is given in Table 4.

TABLE 4
Growth rate of chickens, g

| | Group | | |
|---|---|---|---|
| | I- | II- | III- |
| Index | control* | experimental* | experimental* |
| Live weight: at | 219 | 226 | 224 |
| the beginning of the | | | |
| experiment | | | |
| at the end of the | 2018 | 2390 | 2274 |
| experiment | | | |
| Absolute gain | 1799 | 2164 | 2050 |
| In % to control group | 100 | 120 | 113 |
| Average daily increase | 60 | 72 | 68 |
| In % to control | 100 | 120 | 113 |

At the end of the experiment, the absolute increase in live weight in the experimental group was 365 g greater than in the animals of the control group. At the same time, the average daily increase in live weight in the control group was 60 g, and in the experimental group—72 g. It should be noted that the results in the III experimental group were lower than in the II experimental group. Therefore, it can be concluded that the feed additive according to embodiments of the invention are more effective than the feed additive according to the closest analogue.

The use of a feed additive with drinking water had a positive effect on metabolic processes, feed palatability, digestibility and assimilability of diet nutrients, which contributed to an increase in live weight gain.

1. A feed additive for farm animals and poultry, comprising:
a biomass and/or a cultural liquid of microalgae Chlorella vulgaris, and
chlorellin in a titer of 10⁸-10¹⁴ CFU per 1 ml of the cultural liquid and/or the biomass.

2. The feed additive of claim 1, wherein the feed additive is configured to be added to drinking water.

3. A method of obtaining a feed additive, comprising:
growing a strain of green unicellular microalgae Chlorella vulgaris in a culture to provide a culture liquid and/or a biomass; and
adding chlorellin to the culture liquid and/or the biomass.

4. The method of claim 3, further comprising separating the culture into the culture liquid and the biomass.

5. The method of claim 3, further comprising concentrating and/or drying the culture liquid and/or the biomass.

6. The method of claim 3, further comprising standardizing the culture liquid and/or the biomass.

7. The method of claim 3, wherein the strain of green unicellular microalgae Chlorella vulgaris is grown on a rocking chair.

8. The method of claim 3, wherein the strain of green unicellular microalgae Chlorella vulgaris is grown on an industrial scale in pools.

9. The method of claim 3, wherein the strain of green unicellular microalgae Chlorella vulgaris is grown at a temperature of 30° C. and/or at a pH of 5.5-5.8.

10. The method of claim 3, wherein the strain of green unicellular microalgae Chlorella vulgaris is grown under conditions including aeration and/or mixing.

11. The method of claim 3, further comprising packaging the feed additive.

12. The method of claim 11, wherein packaging the feed additive includes packaging the feed additive for subsequent mixing into drinking water.

13. The method of claim 4, wherein separating the culture into the culture liquid and the biomass is performed by filtration and/or by centrifugation.

14. The method of claim 13, wherein filtration includes a suction filter.

15. The method of claim 3, wherein chlorellin is added in a titer of 10⁸-10¹⁴ CFU per 1 ml of the cultural liquid and/or the biomass.

16. A method of supplementing animal feed, comprising:
providing a feed additive having microalgae Chlorella vulgaris and chlorellin.

17. The method of claim 16, wherein the microalgae Chlorella vulgaris is provided as a biomass and/or a cultural liquid.

18. The method of claim 16, further comprising:
adding the feed additive to drinking water through medicators and/or dispensers.

19. The method of claim 18, wherein the feed additive is mixed with the drinking water in a ratio of approximately 80 ml per 1,000 liters of the drinking water.

20. The method of claim 16, wherein chlorellin is included in a titer of 10⁸-10¹⁴ CFU per 1 ml of the biomass and/or the cultural liquid.
